(19) United States
(12) Patent Application Publication
(10) Pub. No.: US 2024/0277527 A1
(43) Pub. Date: Aug. 22, 2024

(54) In-ear earplug structure

(71) Applicant: Shenzhen Taipuda Technology Co., Ltd., Shenzhen (CN)

(72) Inventors: Dehua Guo, Shenzhen (CN); Jinniu Yuan, Shenzhen (CN)

(21) Appl. No.: 18/648,455

(22) Filed: Apr. 28, 2024

Related U.S. Application Data
(63) Continuation of application No. 17/238,234, filed on Apr. 23, 2021.

(51) Int. Cl. A61F 11/08

(52) U.S. Cl. CPC A61F 11/08; A61F 11/085

(57) ABSTRACT

The present disclosure discloses an in-ear earplug structure, which comprises a hollow support body, wherein a sound hole is opened at a front end of the support body, an outer side of the support body is provided with at least two soft umbrella-shaped plug bodies, the support body and the umbrella-shaped plug bodies are integrally molded, a speaker unit is provided in the support body, a preset spacing is formed between the speaker unit and the sound hole, a rear sound cavity is formed in the support body, the rear sound cavity is located on the rear side of the speaker unit, and a through hole which is in communication with the rear sound cavity is opened at a side part of the support body. In the above structure, when the earplug is worn on an ear, the hard feeling caused by the earplug is greatly reduced, and the wearing comfort is effectively improved. Also, this makes the umbrella-shaped plug body better fit an ear canal, thereby improving the sound insulation effect. In addition, the support body and the umbrella-shaped plug bodies can be integrally processed and molded, making the earplug easier to manufacture and low in cost, which satisfies the needs of users and the market well.

### CROSS-REFERENCE TO RELATED APPLICATIONS

This application is a Continuation of the U.S. application Ser. No. 17/238,234 filed on Apr. 23, 2021, and entitled “In-ear earplug structure”, now pending, the entire disclosures of which are incorporated herein by reference.

### TECHNICAL FIELD

The present disclosure relates to an earplug product, and in particular to an in-ear earplug structure.

### BACKGROUND ART

Earplugs are generally used in earphones or noise-proof products. The structure of an existing in-ear earplug is generally to set two or three umbrella-shaped rubber sleeves on a hard cylindrical tube. With the matching of the umbrella-shaped rubber sleeves and ears, noise reduction function is realized, but because the cylindrical tube at the center is generally made of hard plastic, when the earplug is inserted into an ear canal, the cylindrical tube will obviously bring a hard feeling, which will cause discomfort to wear, especially when used for a long time, the discomfort is more obvious. In addition, such a structure of combination of hard parts and soft parts has an unsatisfactory sound insulation effect, so it cannot meet the noise reduction requirements.

### SUMMARY

The technical problem to be solved by the present disclosure is to provide an in-ear earplug structure that is comfortable to wear, better in sound insulation, easy to manufacture, and low in cost, in view of the deficiencies of the prior art.

In order to solve the above technical problem, the technical solution used by the present disclosure is as follows.

An in-ear earplug structure comprises a hollow support body, wherein a sound hole is opened at a front end of the support body, an outer side of the support body is provided with at least two soft umbrella-shaped plug bodies, the support body and the umbrella-shaped plug bodies are integrally molded, a speaker unit is provided in the support body, a preset spacing is formed between the speaker unit and the sound hole, a rear sound cavity is formed in the support body, the rear sound cavity is located on the rear side of the speaker unit, and a through hole which is in communication with the rear sound cavity is opened at a side part of the support body.

Preferably, the support body and the umbrella-shaped plug bodies are molded by injection using soft rubber materials.

Preferably, the cross section of the umbrella-shaped plug body is oval.

Preferably, two umbrella-shaped plug bodies are formed on the outer side of the support body, and the sizes of the two umbrella-shaped plug bodies increase sequentially from front to back.

Preferably, an inner wall of the support body is formed with an inwardly protruding annular flange.

In the in-ear earplug structure disclosed by the present disclosure, the support body and the umbrella-shaped plug bodies are arranged as an integral structure, such that the main part of the earplug is a soft structure. Meanwhile, the position of the speaker unit is moved back, such that there is a certain distance between the speaker unit and the sound hole. Based on the above structure, when the earplug is worn on an ear, the hard feeling caused by the earplug is greatly reduced, and the wearing comfort is effectively improved. Also, this makes the umbrella-shaped plug body better fit an ear canal, thereby improving the sound insulation effect. In addition, the support body and the umbrella-shaped plug bodies can be integrally processed and molded, making the earplug easier to manufacture and low in cost, which satisfies the needs of users and the market well.

### BRIEF DESCRIPTION OF THE DRAWINGS

FIG. 1 is a first three-dimensional view of the in-ear earplug structure of the present disclosure;

FIG. 2 is a second three-dimensional view of the in-ear earplug structure of the present disclosure;

FIG. 3 is a third three-dimensional view of the in-ear earplug structure of the present disclosure;

FIG. 4 is a cross-sectional view of the in-ear earplug structure of the present disclosure; and

FIGS. 5 and 6 are respectively cross-sectional views of other embodiments of the in-ear earplug structure of the present disclosure.

### DETAILED DESCRIPTION OF EMBODIMENTS

The following describes the present disclosure in more detail with reference to the drawings and embodiments.

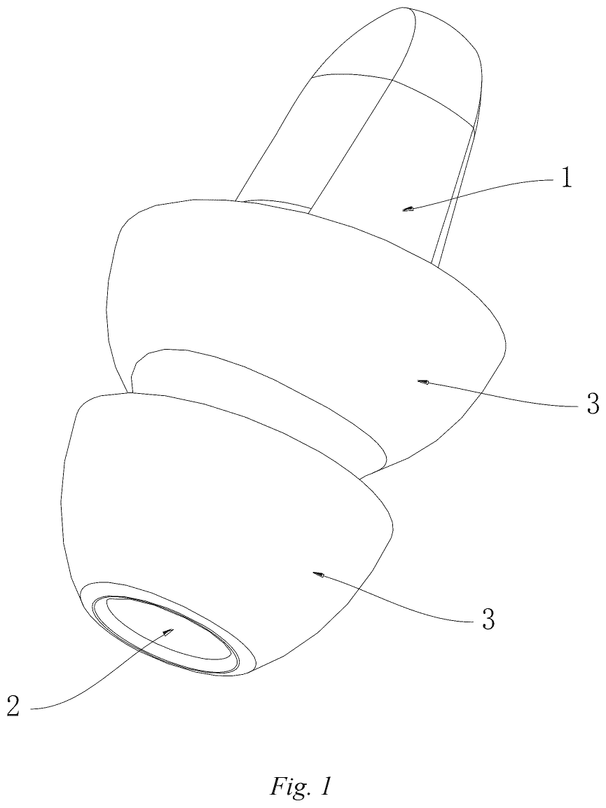

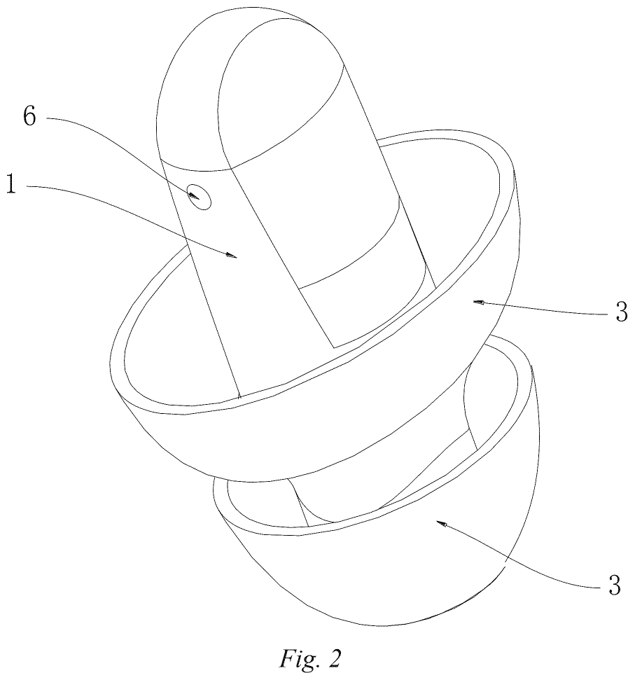

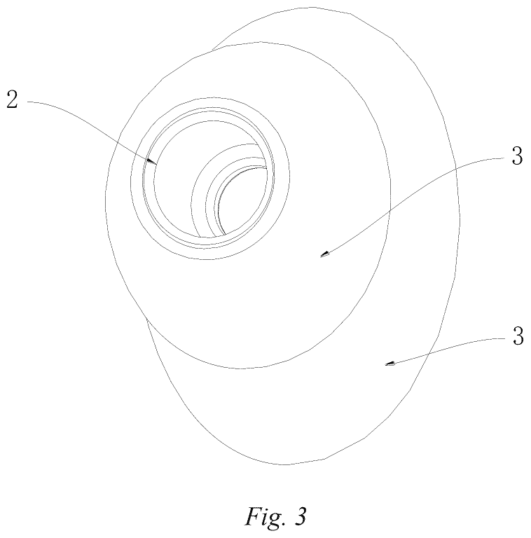

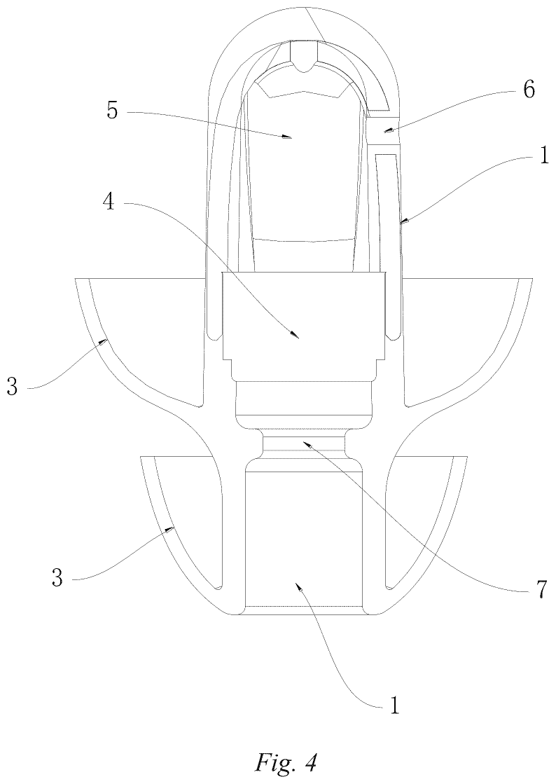

The present disclosure discloses an in-ear earplug structure. As shown in FIG. 1 to FIG. 4, the in-ear earplug structure comprises a hollow support body 1, wherein a sound hole 2 is opened at a front end of the support body 1, an outer side of the support body 1 is provided with at least two soft umbrella-shaped plug bodies 3, the support body 1 and the umbrella-shaped plug bodies 3 are integrally molded, a speaker unit 4 is provided in the support body 1, a preset spacing is formed between the speaker unit 4 and the sound hole 2, a rear sound cavity 5 is formed in the support body 1, the rear sound cavity 5 is located on the rear side of the speaker unit 4, and a through hole 6 which is in communication with the rear sound cavity 5 is opened at a side part of the support body 1.

In the above structure, the support body 1 and the umbrella-shaped plug bodies 3 are arranged as an integral structure, such that the main part of the earplug is a soft structure. Meanwhile, the position of the speaker unit 4 is moved back, such that there is a certain distance between the speaker unit 4 and the sound hole 2. Based on the above structure, when the earplug is worn on an ear, the hard feeling caused by the earplug is greatly reduced, and the wearing comfort is effectively improved. Also, this makes the umbrella-shaped plug body 3 better fit an ear canal, thereby improving the sound insulation effect. In addition, the support body 1 and the umbrella-shaped plug bodies 3 can be integrally processed and molded, making the earplug easier to manufacture and low in cost, which satisfies the needs of users and the market well.

In order to facilitate production and manufacturing, in this embodiment, the support body 1 and the umbrella plug bodies 3 are molded by injection using soft rubber materials.

In this embodiment, the cross section of the umbrella-shaped plug body 3 is oval. The oval structure can make the umbrella plug body 3 better fit the user's ear canal, thereby further improving the sound insulation and noise reduction effect.

As a preferred structure, two umbrella-shaped plug bodies 3 are formed on the outer side of the support body 1, and the sizes of the two umbrella-shaped plug bodies 3 increase sequentially from front to back.

In this embodiment, an inner wall of the support body 1 is formed with an inwardly protruding annular flange 7. The annular flange 7 can strengthen the support body 1 internally.

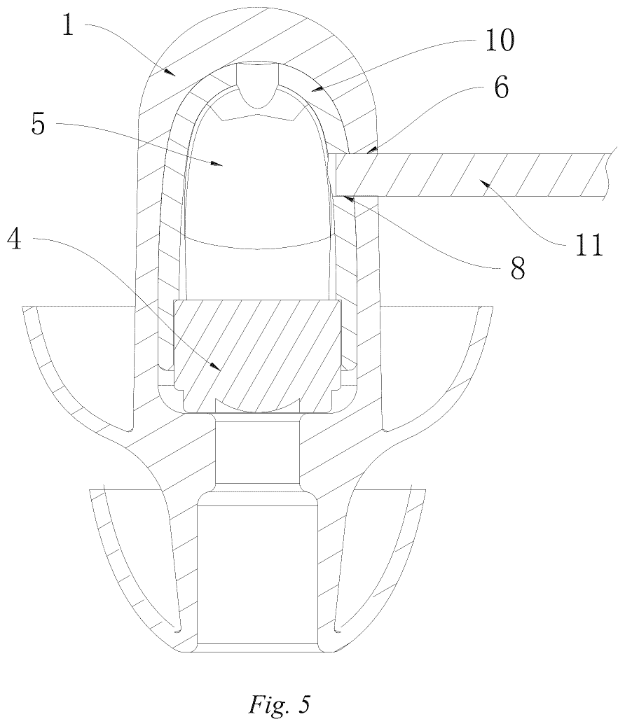

In other embodiments, as shown in FIG. 5, the rear sound cavity 5 is an inner cavity of an inner shell 10 embedded in the support body 1 and entirely wrapped by the support body 1. Moreover, the speaker unit 4 is inserted into one end of the inner shell 10, a wire hole 8 communicating with the through hole 6 of the support body 1 is provided on a side of the inner shell 10, mainly for a wire harness 11 to pass through. The inner shell 10 is made of hard plastic or metal materials. Such a structure can ensure that the shape and size of the rear sound cavity 5 will not change, so as to improve the sound quality. In addition, since the support body 1 is made of soft rubber, the inner shell 10 is entirely wrapped by the support body 1, and the inner shell 10 cannot be easily pulled out directly by the wire harness, which ensures the stability of the inner shell 10 inside the support body 1.

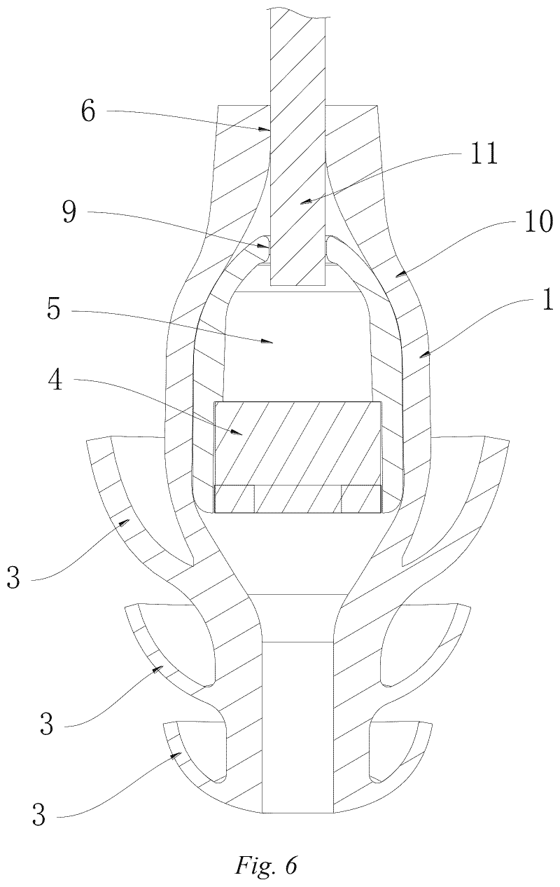

In other embodiments, as shown in FIG. 6, three umbrella-shaped plug bodies 3 are provided on the outer side of the support body 1, and the sizes of the three umbrella-shaped plug bodies 3 increase sequentially from front to back (from bottom to top in FIG. 6). The rear sound cavity 5 is an inner cavity of an inner shell 10 embedded in the support body 1 and entirely wrapped by the support body 1. Moreover, the speaker unit 4 is inserted into one end of the inner shell 10, a wire hole 9 communicating with the through hole 6 of the support body 1 is provided on another end of the inner shell 10, mainly for a wire harness 11 to pass through. The inner shell 10 is made of hard plastic or metal materials. Such a structure can ensure that the shape and size of the rear sound cavity 5 will not change, so as to improve the sound quality. In addition, since the support body 1 is made of soft rubber, the inner shell 10 is entirely wrapped by the support body 1, and the inner shell 10 cannot be easily pulled out directly by the wire harness, which ensures the stability of the inner shell 10 inside the support body 1.

The above embodiments are merely preferred embodiments of the present disclosure but not intended to limit the present disclosure, and any modifications, equivalent replacements, improvements, etc. made within the spirit and principle of the present disclosure should be included within the scope of protection of the present disclosure.

1. An in-ear earplug structure, characterized by comprising a hollow support body (1), wherein a sound hole (2) is opened at a front end of the support body (1), an outer side of the support body (1) is provided with at least two soft umbrella-shaped plug bodies (3), the support body (1) and the umbrella-shaped plug bodies (3) are integrally molded, a speaker unit (4) is provided in the support body (1), a preset spacing is formed between the speaker unit (4) and the sound hole (2), a rear sound cavity (5) is formed in the support body (1), the rear sound cavity (5) is located on the rear side of the speaker unit (4), and a through hole (6) which is in communication with the rear sound cavity (5) is opened at a side part of the support body (1).

2. The in-ear earplug structure of claim 1, characterized in that the support body (1) and the umbrella-shaped plug bodies (3) are molded by injection using soft rubber materials.

3. The in-ear earplug structure of claim 1, characterized in that the cross section of the umbrella-shaped plug body (3) is oval.

4. The in-ear earplug structure of claim 1, characterized in that two umbrella-shaped plug bodies (3) are formed on the outer side of the support body (1), and the sizes of the two umbrella-shaped plug bodies (3) increase sequentially from front to back.

5. The in-ear earplug structure of claim 1, characterized in that an inner wall of the support body (1) is formed with an inwardly protruding annular flange (7).

6. The in-ear earplug structure of claim 1, characterized in that three umbrella-shaped plug bodies (3) are provided on the outer side of the support body (1), and the sizes of the three umbrella-shaped plug bodies (3) increase sequentially from front to back.

7. The in-ear earplug structure of claim 1, characterized in that the rear sound cavity (5) is an inner cavity of an inner shell (10) embedded in the support body (1) and entirely wrapped by the support body (1), and the speaker unit (4) is inserted into one end of the inner shell (10).
